(19) United States
(12) Patent Application Publication
(10) Pub. No.: US 2011/0046716 A1
(43) Pub. Date: Feb. 24, 2011

(54) STENT

(75) Inventors: Richard John Parkinson, Darlinghurst (AU); Zoran Milijasevic, Bayview (AU)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: MURRAY VASCULAR PTY LIMITED, Mosman, NSW (AU)

(21) Appl. No.: 12/867,745

(22) PCT Filed: Feb. 20, 2009
(86) PCT No.: PCT/AU2009/000198

§ 371 Date: Nov. 4, 2010

Related U.S. Application Data
(60) Provisional application No. 61/064,178, filed on Feb. 20, 2008.

(51) Int. Cl. A61F 2/82

(52) U.S. Cl. 623/1.15

(57) ABSTRACT

An endovascular stent, including: a guide portion and a drive portion; a plurality of flexible support arms, each including two opposing ends that are coupled to said guide portion and said drive portion respectively, said arms being moveable relative to each other between an expanded position and a compressed position; and said stent including at least one barrier portion, such that when said arms are moved to said expanded position, said arms configure the barrier portion into a selectively permeable barrier through which one or more articles may be introduced into a space between said barrier and a wall of a lumen receiving said stent.

### FIELD

The present invention relates to endovascular stents, and in particular, but not being limited to, adjustable and retrievable endovascular stents.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

An important development in neurovascular medicine has been the ability to treat defects in relatively small arteries and veins, such as those in the neurovascular system, by use of a guiding catheter and the placement of embolic coils (or the like) in areas where an aneurysm is likely to cause (or has already caused) a rupture in the blood vessel. When the aneurysm is in the brain, it is often difficult to treat small defects in the blood vessels with conventional surgical techniques.

One aspect of these surgical treatments is that an aneurysm (or other malformation) is symptomatic of a general weakening of the vasculature in the area containing the aneurysm. Mere treatment of the aneurysm does not necessarily prevent a subsequent rupture in the surrounding area of the vessel. When a vaso-occlusive device (e.g. an embolic coil) is placed in an aneurysm, it can be difficult to prevent the migration of these small devices away from the aneurysm, particularly where the aneurysm has a relatively large neck to dome ratio. If such migration occurs, the vaso-occlusive device may cause a blockage at another part of a blood vessel, which could result in the patient having a stroke.

Stents, which are typically tubular reinforcements inserted into a blood vessel to provide an open path within the blood vessel, have been widely used in intravascular angioplasty treatment of narrowed cardiac arteries. A stent may be inserted after an angioplasty procedure in order to prevent restenosis of the artery. In such applications, stents are often deployed by use of inflatable balloons, or mechanical devices which force the stent open, thereby reinforcing the artery wall in the clear through-path in the centre of the artery after the angioplasty procedure to prevent restenosis. Although such procedures may be useful in certain aspects of vascular surgery in which vaso-occlusive devices are used, the weakness of the vasculature and the inaccessibility of the interior of the aneurysm from the vessel after the placement of such a stent, places limits on the applicability of such stents in procedures to repair aneurysms, particularly cerebral aneurysms. Furthermore, the use of placement techniques, such as balloons or mechanical expansions (e.g. of the type often found to be useful in cardiac surgery) are less useful and more dangerous (e.g. easier to cause damage or rupture) when such devices are used to treat more fragile vessels (e.g. those found in the brain).

Stenting of the intracranial circulation requires formal anticoagulation and antiplatelet therapy to maintain patency for a permanent endoprosthesis. Stenting in the intracranial circulation may be performed for unruptured cerebral aneurysms to allow reinforcement of the aneurysm neck to allow coiling of an anatomically unfavourable (e.g. a poor dome to neck ratio) aneurysm. In the case of a ruptured aneurysm, the anticoagulation and antiplatelet therapy required to maintain patency of the stent would expose the patient to an unacceptably high risk of death with a further rupture, and stenting is therefore generally not desirable for acutely ruptured aneurysms for this reason. Another device which can be used is a compliant balloon (so called balloon remodelling technique), but this device necessitates occlusion of the parent vessel with a risk of embolism and stroke.

It is desired to address one or more of the above problems, or to at least provide a useful alternative to existing stents.

### SUMMARY

According to the present invention, there is provided an endovascular stent, including:

a guide portion and a drive portion;

a plurality of flexible support arms, each including two opposing ends that are coupled to said guide portion and said drive portion respectively, said arms being moveable relative to each other between an expanded position and a compressed position; and

said stent including at least one barrier portion, such that when said arms are moved to said expanded position, said arms configure the barrier portion into a selectively permeable barrier through which one or more articles may be introduced into a space between said barrier and a wall of a lumen receiving said stent.

The present invention also provides a method for introducing a vaso-occlusive device into an aneurysm of a lumen using a stent (as described above), including:

i) positioning said stent in said lumen adjacent to said aneurysm;

ii) adjusting said arms to said expanded position to form the permeable barrier having one or more an openings adjacent to a neck of said aneurysm; and

iii) delivering said device through the openings into said aneurysm;

wherein, after said device is released into said aneurysm, said barrier inhibits movement of said device away from said aneurysm.

### BRIEF DESCRIPTION OF THE DRAWINGS

Representative embodiments of the present invention are herein described, by way of example only, with reference to the accompanying drawings, wherein:

FIG. 1 is a perspective view of a deployed stent according to one embodiment;

FIG. 2 is a side view of the stent shown in FIG. 1;

FIG. 3 is a detailed side view of the stent shown in FIG. 1;

FIG. 4 is a sectional view of the stent along section A-A in FIG. 3;

FIG. 5 is a perspective view of a stent shown in FIG. 1 without a barrier member;

FIG. 6 is a side view of the stent shown in FIG. 5;

FIG. 7 is a diagram of the stent shown in FIG. 1 during use;

FIG. 8 is a side view of the stent in FIG. 1 in a partially deployed configuration;

FIG. 9 is a detailed side view of the stent shown in FIG. 8;

FIG. 10 is a sectional view of the stent along section B-B in FIG. 9;

FIG. 11 is a perspective view of the stent shown in FIG. 8;

FIGS. 12, 13 and 14 are end, side and perspective views of a deployed stent with six support arms;

FIGS. 15, 16, and 17 are end, side and perspective views of a deployed stent with eight support arms;

FIGS. 18 and 19 are side and perspective views of a deployed stent with multiple barrier members;

FIGS. 20 and 21 are side and perspective views of the stent shown in FIG. 5 in a collapsed configuration;

FIGS. 22, 23 and 24 are end, side and perspective views of a deployed stent with a reinforced support section;

FIGS. 25, 26, and 27 are end, side and perspective views of a deployed stent with a partially reinforced support section;

FIG. 28 is a sectional view of the stent along section D-D in FIG. 2;

FIG. 29 is a sectional view of the barrier member of the stent shown in FIG. 28;

FIG. 30 is a sectional view of the pocket corresponding to Detail E of FIG. 28;

FIGS. 31, 32 and 33 are end, side and perspective views of a barrier portion of another representative embodiment of the stent;

FIGS. 34 to 41 are diagrams relating to a representative embodiment of the stent;

FIGS. 42 to 45 are diagrams relating to a representative embodiment of the stent.

### DETAILED DESCRIPTION OF THE REPRESENTATIVE EMBODIMENTS

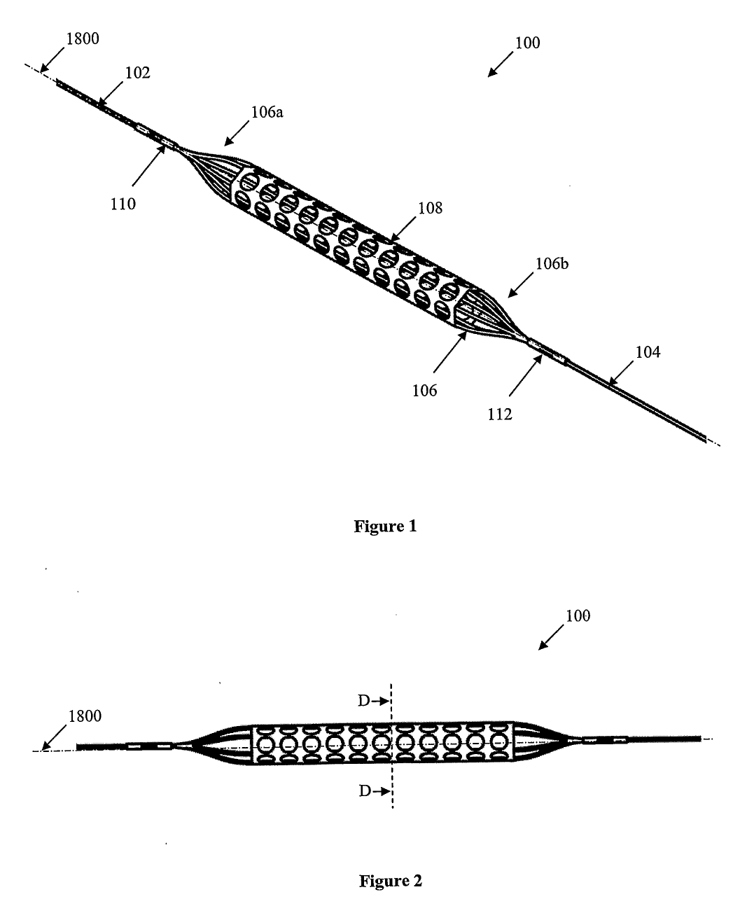

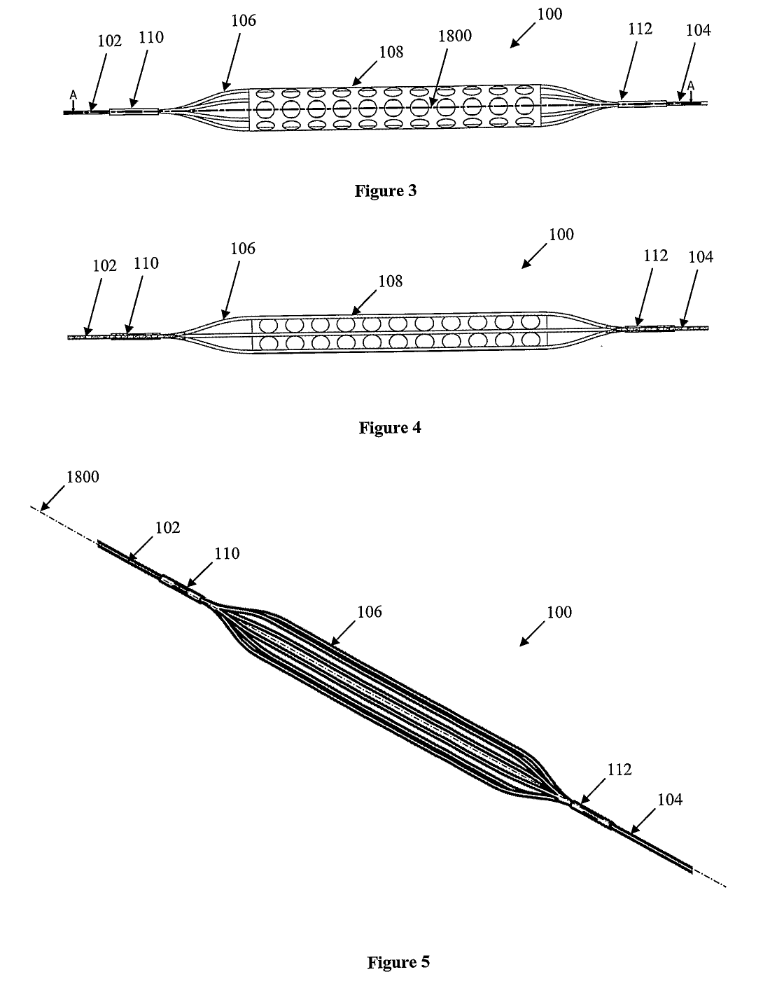

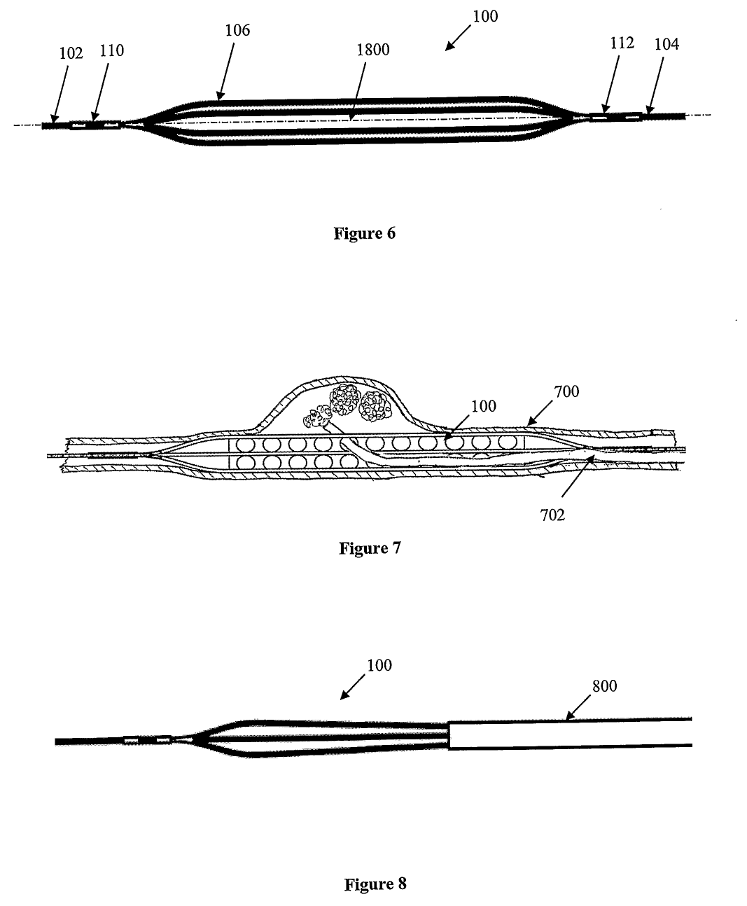

A representative embodiment of an adjustable endovascular stent 100, as shown in FIG. 1, includes a guide portion 102, a drive portion 104, and a plurality of flexible support arms 106 arranged about a longitudinal axis 1800 of the stent 100. The drive portion 104 may include a flexible drive member (e.g. a microwire) for controlling the lateral movement or positioning of the stent 100 within a body lumen (e.g. a blood vessel). Alternatively, the drive portion 104 may be adapted so that it can be releasably coupled to a flexible drive member. The guide portion 102 may include a flexible guide member (e.g. a microwire) for guiding the directional movement of the stent 100 when pushed along the lumen (e.g. by the flexible drive member).

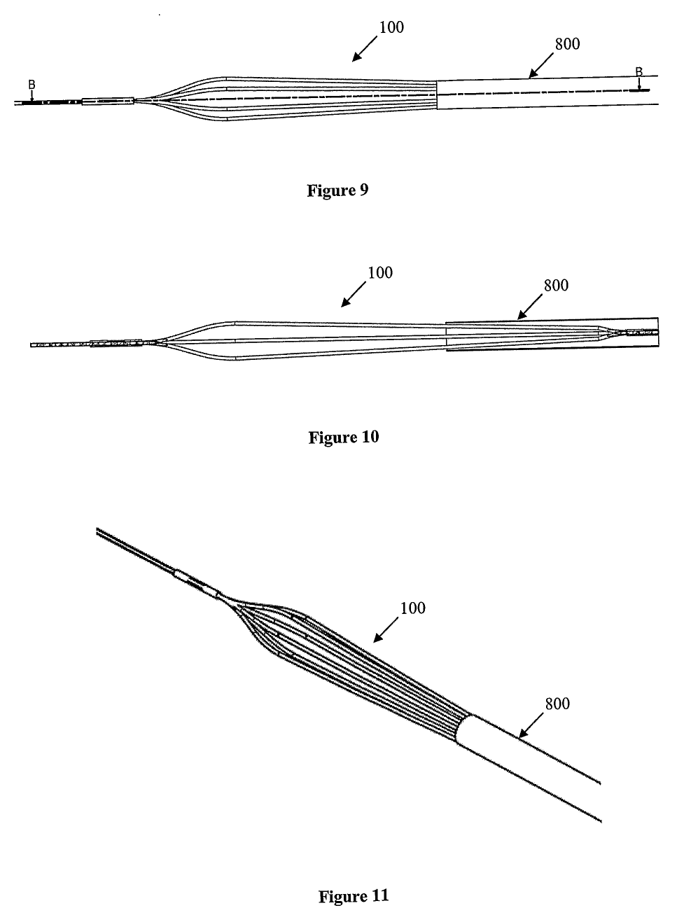

Each of the support arms 106 has two opposing end portions 106a and 106b that are coupled to the guide portion 102 and drive portion 104 respectively. The support arms 106 are moveable relative to each other between an expanded position and a compressed position. FIG. 1 shows a representative embodiment of the stent 100 with its support arms 106 configured in a fully expanded (i.e. deployed) position. The support arms 106 are arranged about a longitudinal axis 1800 of the stent 100. Each of the support arms 106 may be shaped so that one or more portions of the arms 106 are arranged in parallel to the axis 1800. The support arms 106 can move away from the axis 1800 to the expanded position, and move towards the axis 1800 to the compressed position. FIGS. 9 to 11 show the stent 100 in a partially deployed position, where a portion of the support arms 106 of the stent 100 are still held in its partially compressed position within a catheter 800. The stent 100 is in a fully compressed position when the stent 100 is wholly received within the catheter 800.

The number of support arms 106 included in different stents 100 (or in the various sections/portions of the stent) may vary depending upon the type of functional characteristics to be provided by a particular stent 100. For example, a stent 100 may have fewer support arms 106 to improve the ability for a fluid (e.g. blood) to flow through the lumen in which the stent 100 is received. Alternatively, a stent 100 may have a greater number of support arms 106 for providing better support of the lumen wall, and/or for providing a more effective barrier that helps inhibit the movement of an article (e.g. a vaso-occulsive device such as an embolic coil) introduced into a space between the stent 100 and the lumen wall. For example, when the support arms 106 are configured to the expanded position, the gaps formed between adjacent arms 106 are smaller than the article so that the position of the arms 106 for an effective barrier for inhibiting the movement of the article away from its position between the stent 100 and the lumen wall.

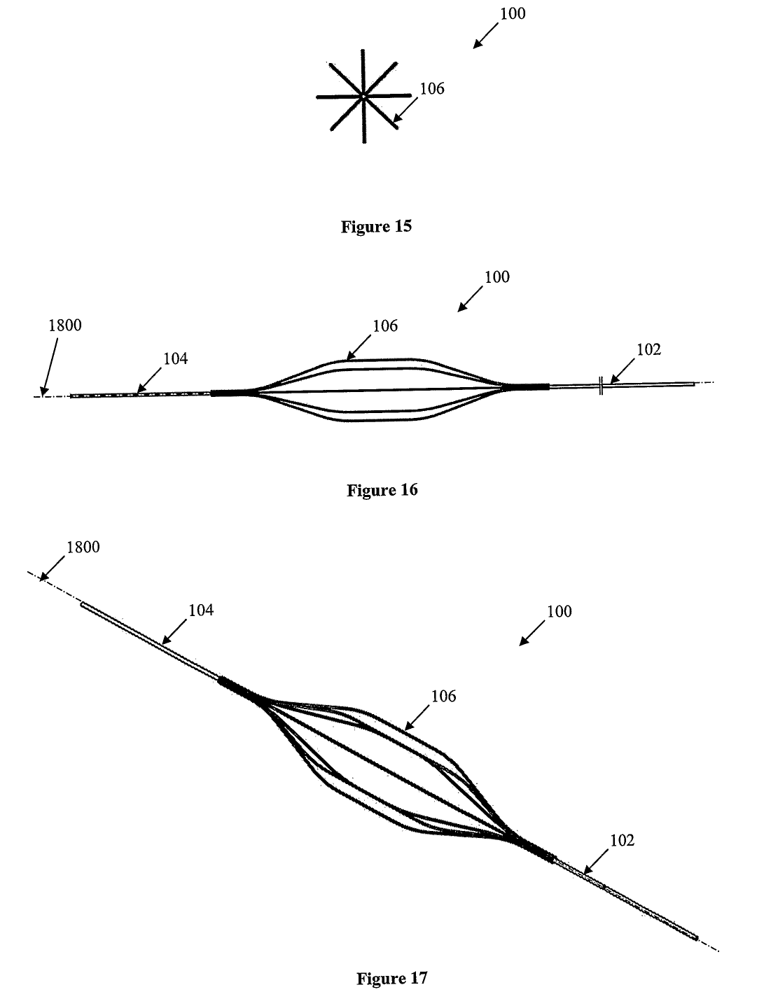

In one representative embodiment, as shown in FIGS. 1, 12, 13 and 14, the stent 100 may have 6 support arms that are radially and evenly spaced from each other about a longitudinal axis 1800 (along which the guide portion 102 and drive portion 104 are aligned). In another embodiment, as shown in FIGS. 15, 16 and 17, the stent 100 may have 8 support arms that are radially and evenly space from each other about a longitudinal axis 1800 (on which the guide portion 102 and drive portion 104 are aligned).

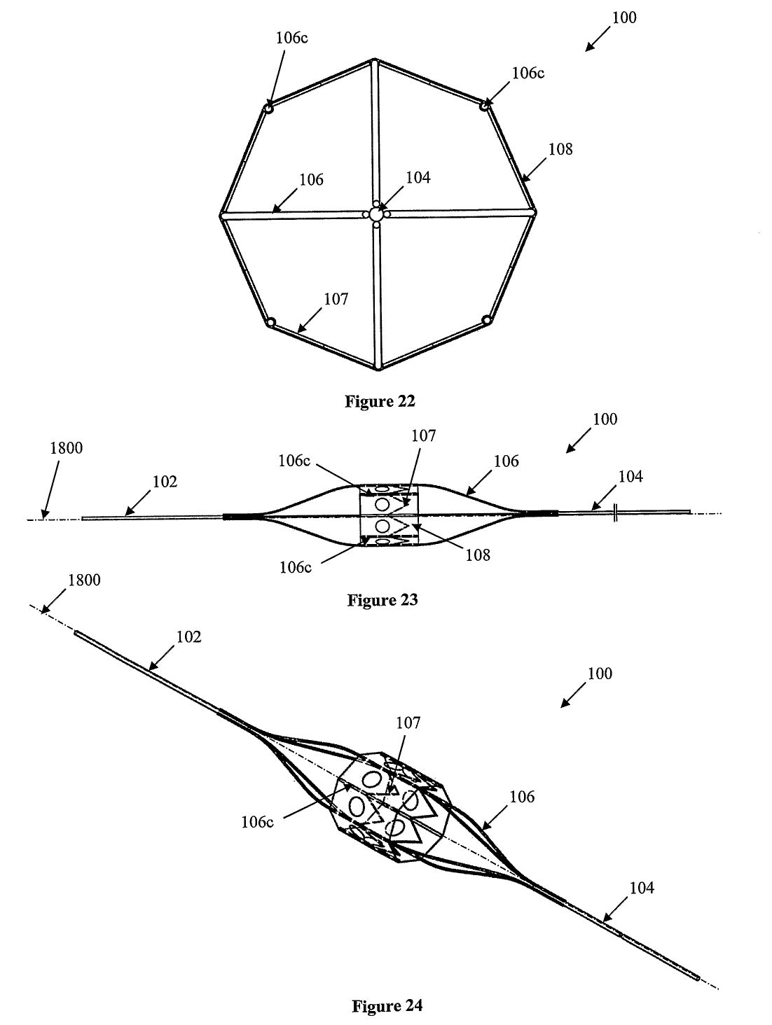

The representative embodiments of the stent 100 shown in FIGS. 22 to 27 have less support arms 106 at the end sections of the stent 100, which provides less obstruction to a flow of fluid (e.g. blood) through the stent 100. These representative embodiments also have additional support arms 106c in a centre section of the stent 100 to provide a better (e.g. more circular) support of a lumen wall, or for defining a barrier portion around a certain section of the stent 100). The barrier portion may include one or more support arms 106 and 106c, or may include an additional barrier member 108 (e.g. a membrane) that surrounds at least some of the support arms 106 and 106c.

FIGS. 22, 23 and 24 respectively show the end view, side view and isometric view of a representative embodiment of a stent 100 having four basic support arms 106 with opposing end portions that are coupled to the guide portion 102 and drive portion 104 respectively. The stent 100 shown in FIGS. 22, 23 and 24 has one or more additional support arms 106c that are coupled to the basic support arms 106 by one or more support structures 107. The additional support arms 106c may be arranged in parallel to the longitudinal axis 1800 of the stent 100. A support structure 107 may include one or more deformable members with a flexible portion (e.g. a foldable or hinged portion) having opposing ends that can move towards or away from each other depending on the configuration of the basic support arms 106. A support structure 107, as shown in FIG. 23, may have a V-shaped configuration. When the support arms 106 are adjusted to an expanded position, the support structures 107 also expand to push the additional support arms 106c radially outwards and away from each other. When the support arms 106 are adjusted to a compressed position, the support structures 107 also compress to allow the additional support arms 106c to move radially inwards and towards each other.

The stent 100 as shown in FIGS. 22, 23 and 24 has a barrier portion 108 that extends radially around all of the support arms 106 and 106c of the stent 100. The barrier portion 108 may include one or more barrier members (e.g. a membrane) that can be made to any length so as to extend along at least a portion of the stent 100. FIG. 22 is an end view of the stent 100 shown in FIG. 23 when viewed from direction C.

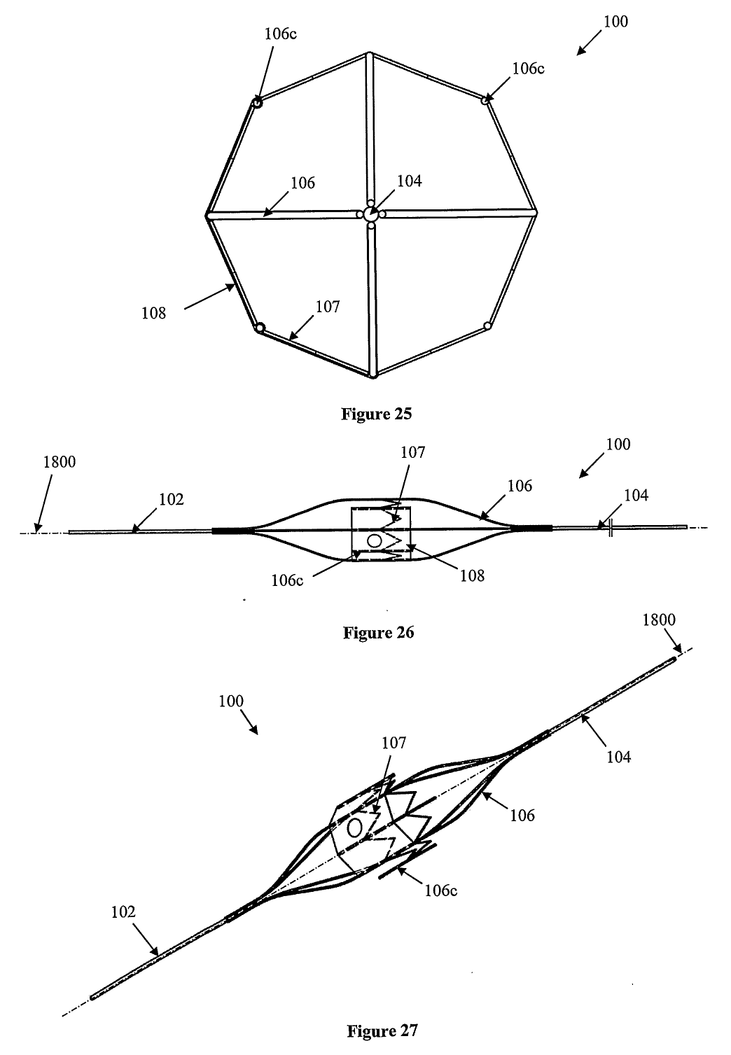

The stent 100 as shown in FIGS. 25, 26 and 27 has a similar configuration of support arms 106 and 106c as that shown in FIGS. 22, 23 and 24, except that the barrier portion 108 only partially extends around some of the support arms 106 and 106c of the stent 100.

In a representative embodiment, the support arms 106 are biased to move towards an expanded position, due to the flexible and spring-like properties of the support arms 106, which may be shaped with a bow-like curvature. The respective ends 106a and 106b of the support arms 106 are securely coupled to the guide portion 102 and drive portion 104 of the stent 100 by different crimp tubes 110 and 112. Alternatively, the support arms 106 can be welded to guide portion 102 and drive portion 104. This coupling configuration holds the ends 106a and 106b of the support arms 106 together and allows a centre portion of the support arms 106 to expand and compress (e.g. in an “umbrella-like” manner).

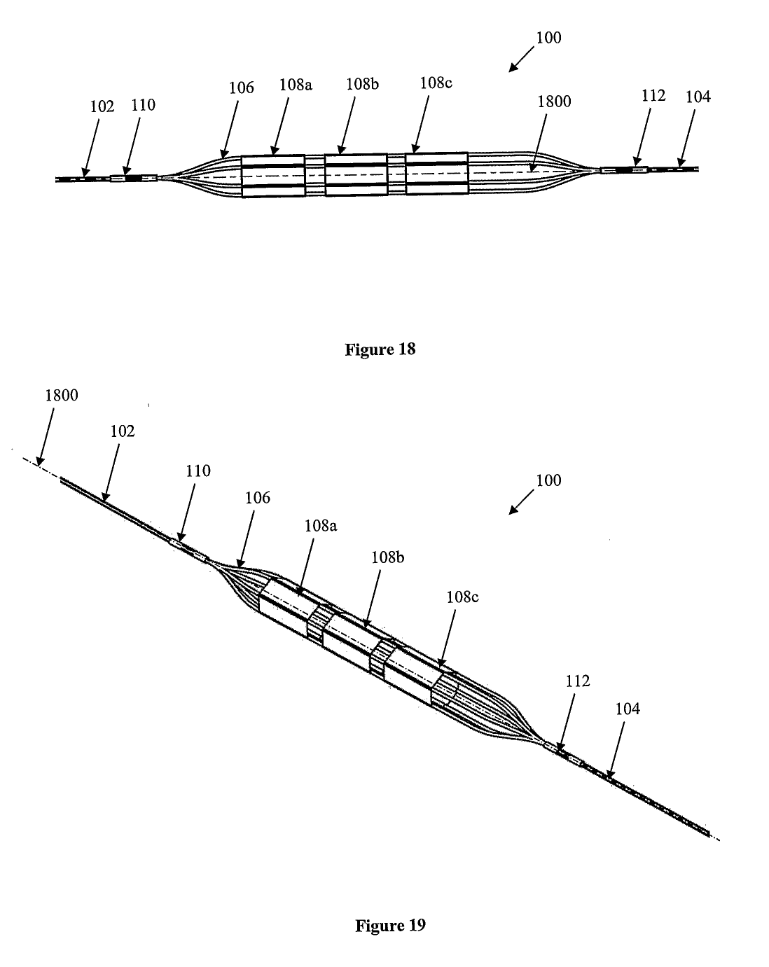

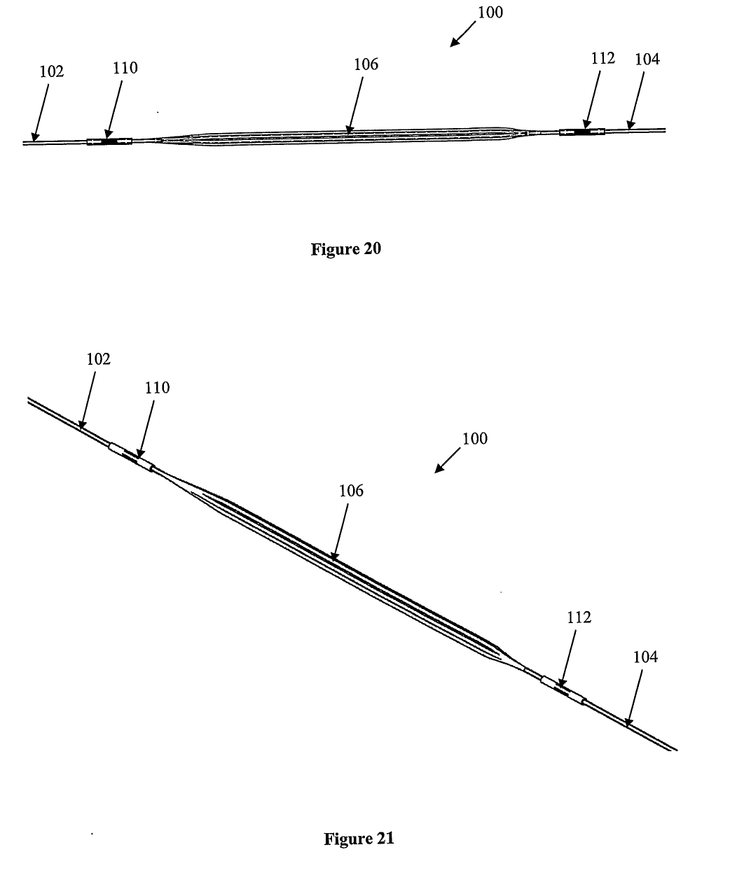

As shown in FIGS. 1 and 18, the support arms 106 of the stent 100 are arranged about a longitudinal axis 1800, where each support arm 106 is substantially linear, and are arranged in parallel to the longitudinal axis 1800. The longitudinal axis 1800 (see FIGS. 1 and 2) may be an axis along which the guide portion 102 and drive portion 104 are aligned. Equally however, in other representative embodiments of the invention, the longitudinal axis 1800 may not be so aligned. For example, in one representative embodiment, the support arms 106 (when placed in the expanded position) may form an eccentric cross-sectional shape where the longitudinal axis 1800 relative to which the arms 106 expand and retract is different from (e.g. is not aligned to) the axis passing between the guide member 102 and drive member 104 of the stent 100.

The support arms 106 are adjustable to move away from the axis 1800 to the expanded position, or alternatively, move towards the axis 1800 to the compressed position. The support arms 106 can have any shape or configuration (e.g. a spiral or helical configuration) which enables the support arms 106 to move between the expanded and compressed positions.

The stent 100 has a barrier portion which may include at least one barrier member 108 that is coupled to the support arms 106. The barrier member 108 may be of any material suitable to form the permeable barrier.

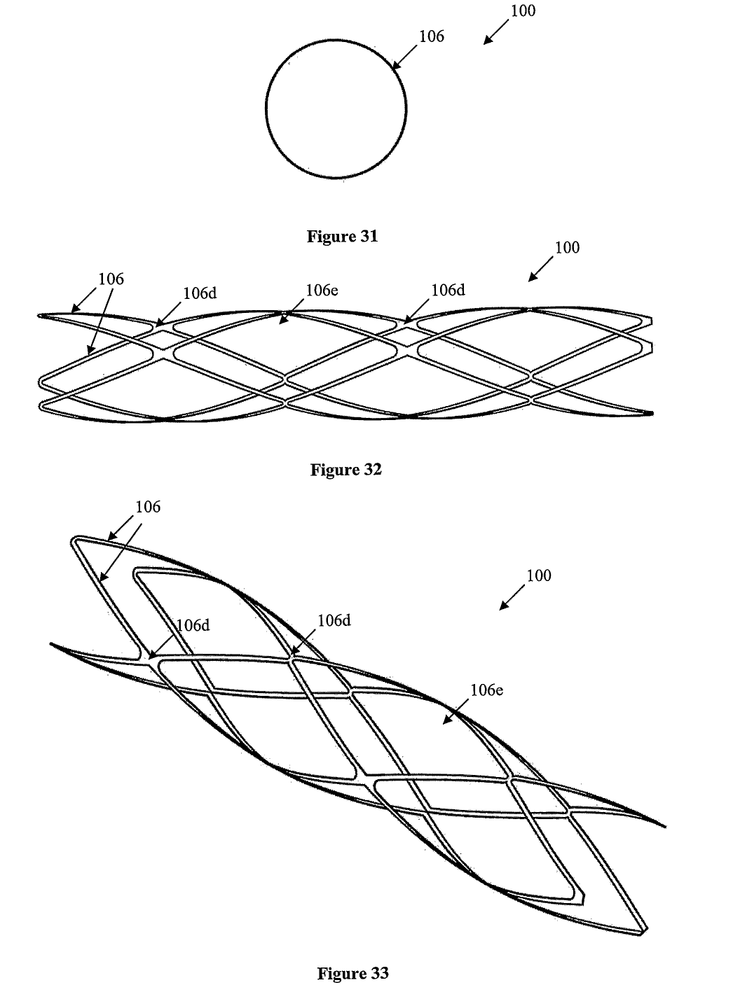

In one representative embodiment, the material of the barrier member 108 may be substantially non-elastic but capable of unfolding and being stretched taut to form a barrier such as occurs in a conventional umbrella arrangement. In another embodiment, the barrier member 108 may be made of a material that is elastic, and therefore can be elastically stretched to form a barrier. In yet another embodiment, the barrier member 108 may be made of a deformable (or plastic) material in the sense that upon being stretched, it may be plastically deformed to form a barrier. In a representative embodiment, the barrier member 108 is a membrane made from an elastomer material (such as silicones, latex and natural and synthetic rubbers), and/or a polymer material (such as polytetrafluoroethylene (PTFE), perfluoroalkoxy polymer resin (PFA), fluorinated ethylene-propylene (FEP), polyethylene, polyurethane). Alternatively, the barrier member 108 could be made from a metallic material (such as a nitinol, nickel or titanium alloy, or another alloy with similar elastically deformable or spring-like properties). The barrier portion of the stent 100 may include a barrier member 108 formed as a flexible or expandable mesh. For example, the barrier portion of the stent may include an adjustable mesh configuration of arms, as shown in FIGS. 31, 32 and 33. The arms in such a mesh configuration overlap (and may be fixed or coupled together) at specific locations intermediate to the arms. Alternatively, the arms in such a mesh configuration are able to slide relative to each other when the barrier member 108 is adjusted between the expanded and compressed positions.

In these arrangements, when the support arms 106 are adjusted to the expanded position, as shown in FIG. 1, the barrier portion of the stent 100 (e.g. including a barrier member 108) is stretched by the arms 106 and are positioned proximate to an inner wall of a body lumen (e.g. a blood vessel). The barrier portion of the stent 100 (e.g. a stretched barrier member 108) forms a permeable barrier that (i) is deformable for allowing one or more articles (e.g. a vaso-occlusive device, such as an embolic coil) to pass through the barrier to be introduced into a space between the barrier and the lumen wall, and (ii) returns to a configuration (after introducing the one or more articles) for inhibiting the movement of the one or more articles away from the space between the barrier and the lumen wall.

The barrier portion of the stent 100 defines one or more openings. For example, as shown in FIG. 1, the openings may be formed through a portion of a barrier member 108, which are shown as circular holes in FIG. 1. Alternatively, the barrier member 108 may include one or more slits that operate in a valve-type arrangement. Each slit is biased so it is normally pursed together in a closed position to form a barrier, and each slit can be pushed apart (e.g. by a penetrating delivery catheter) to define an opening that allows objects to pass through the barrier member 108. The barrier member 108 can be a mesh tube with open tines or holes that are anchored to the drive member 104 by the arms 106.

In the embodiment shown in FIGS. 18 and 19, the stent 100 has a plurality of barrier members 108a, 108b and 108c, and the openings are defined by the gaps between adjacent barrier members 108a, 108b and 108b.

In the embodiment shown in FIGS. 25, 26 and 27 the barrier member 108 only partially extends radially around the stent 100. In this arrangement the defined opening will permit introduction of a vaso-occlusive device into the site of an aneurysm. The stent 100 can then be rotated to present the barrier member 108 to the site to retain the articles between the barrier member 108 and the lumen wall 700.

In a separate embodiment of the invention, the barrier member 108 may comprise a plurality of additional arms 106 selectively positioned in the central section of the stent 100 which together form the desired barrier with openings between those arms. An example of this embodiment is the stent 100 shown in FIGS. 22, 23 and 24 without the barrier member 108.

FIGS. 31, 32 and 33 respectively show an end view, side view and perspective view of an example of an expandable barrier portion of a stent 100 formed by a plurality of support arms 106 in an mesh configuration. For example, the support arms 106 may have a spiral shape, and may overlap with each other (e.g. in an interwoven configuration) to form a mesh. In a representative embodiment, the support arms 106 overlap at different positions 106d (as shown in FIG. 32), and are joined or coupled together at these positions 106d. In this configuration, the support arms 106 move away from each other to define openings 106e (e.g. of a diamond shape as shown in FIG. 32) that is adjustable in size (up to a fixed size) that serve the same function as the openings of a barrier member 108. Alternatively, the support arms 106 are not joined together at the overlapping positions 106d, and support arms 106 can slide over (or relative to) each other when the stent 100 is adjusted to the expanded or compressed positions. In this configuration, the size of the openings will depend on the position of one support arm 106 relative to other adjacent support arms 106.

Preferably, the openings of the barrier portion of the stent 100 are adjustable to a sufficiently large size in order to provide access to the space between the barrier and the lumen wall to enable the delivery of one or more articles (e.g. vaso-occlusive devices) in this space. The openings should also be adjustable to a size that is sufficiently small for inhibiting the one or more articles retained between the barrier and the lumen wall from moving away from this space once released into the space.

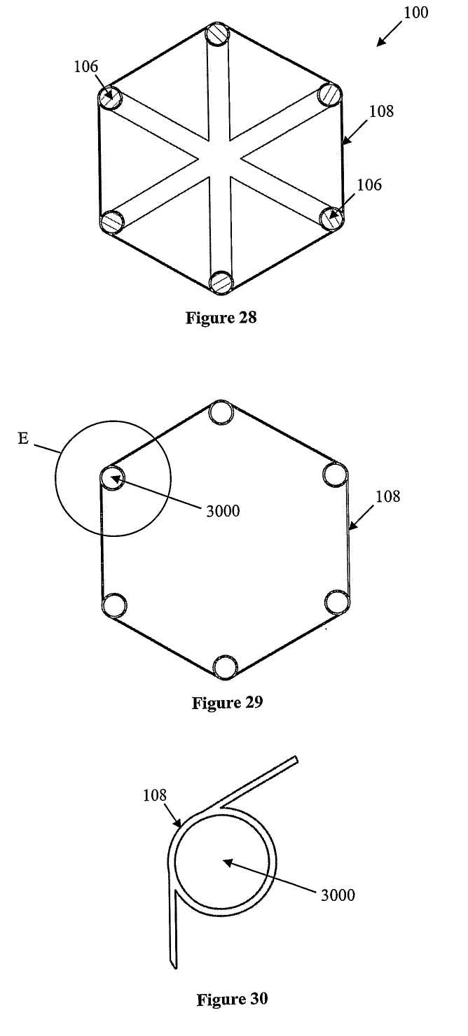

A barrier member 108 may include one or more preformed pockets 3000 for receiving and engaging a portion a particular support arm 106, as shown in FIGS. 28 and 29. FIG. 28 is a cross-section view of the stent 100 along section D-D of FIG. 2. FIG. 29 is a cross-sectional view of only the barrier member 108 of the stent portion shown in FIG. 28. FIG. 30 is a detailed view of a pocket 3000 corresponding to Detail E in FIG. 29. Each of the pockets provides a tight fit with a support arm 106, so that the friction between the support pockets and support arms 106 minimises the movement of the barrier member 108 relative to the arms 106. For example, the friction from the pockets prevents the barrier member 108 from falling off the stent 100, such as when the barrier member 108 is rubbed against a section of a lumen wall when the stent 100 is fully expanded in the lumen.

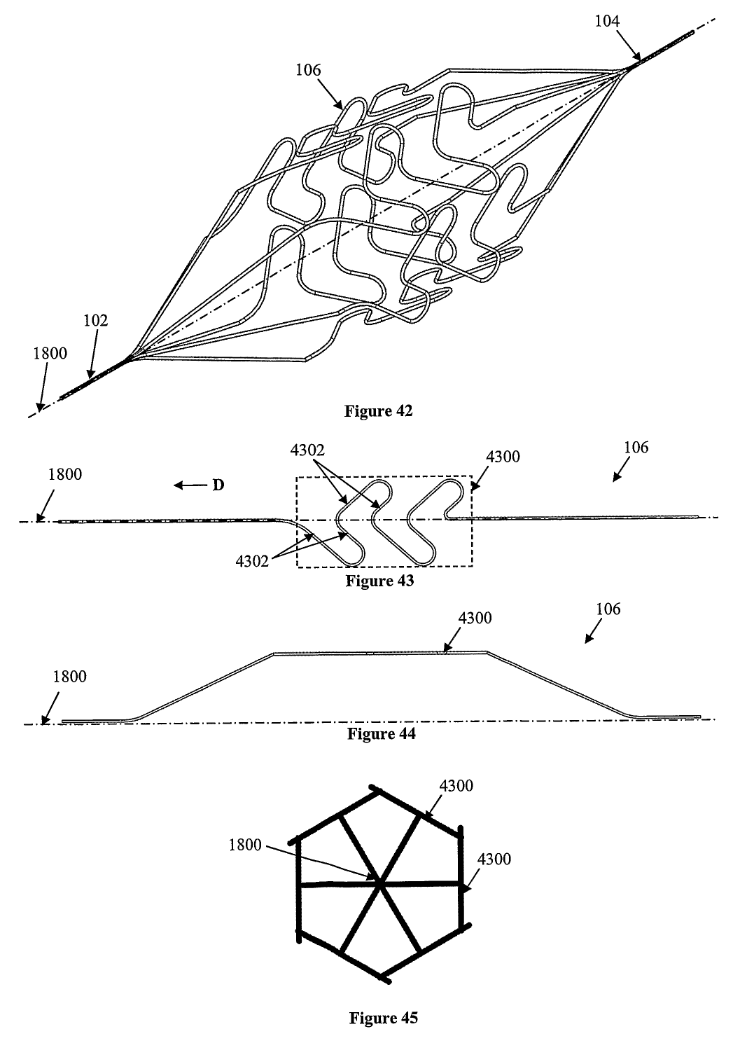

FIGS. 42 to 45 are diagrams relating to another representative embodiment of the stent 100. The stent 100, as shown in FIG. 42, includes a plurality of support arms 106 arranged about a longitudinal axis 1800. The respective ends of each of the support arms 106 are coupled to a guide portion 102 and a drive portion 104. The drive portion 104 of the stent 100 may be releasably coupled to a drive member (not shown in FIG. 42).

FIGS. 43 and 44 are top and side views of a single support arm 106 for use in a stent 100 as shown in FIG. 42. As shown in FIG. 44, each support arm 106 is shaped (e.g. with a bow-like curvature from the side) for biasing a support portion of the support arm 106 away from the longitudinal axis 1800 for configuring the stent 100 in the expanded position. The support portion of the support arm 106 may be pushed towards the longitudinal axis 1800 (e.g. when the stent 100 is received into a catheter) for configuring the stent 100 in the compressed position.

As shown in FIG. 43, each support arm 106 includes a portion that is shaped for defining a deformable barrier structure 4300. The barrier structure 4300 can take any shape or form, and for example, may advantageously have a substantially sinusoidal shape as shown in FIG. 43. The barrier structure 4300 may also be formed along a plane that is substantially normal to the direction in which the support arm 106 moves towards or away from the longitudinal axis 1800. In the representative embodiment shown in FIG. 43, the body of a support arm 106 is shaped to include a barrier structure 4300 that includes one or more adjustable barrier arm portions 4300 formed on the plane (described above).

The barrier arm portions 4300 may each be biased towards an angled position relative to the longitudinal axis 1800. Accordingly, when the support arm 106 moves in a direction indicated by direction arrow D in FIG. 43 (e.g. to be received into a catheter for storage), the barrier arm portions 4300 can be forced to move along the plane in a direction towards the longitudinal axis 1800 so that the barrier arm portions 4300 adopt a compressed configuration. Such force may be applied by the wall of a catheter for receiving the stent 100. In the absence of such force (e.g. when the stent 100 is released from the catheter), the barrier arm portions 4300 are able to move to an expanded configuration as shown in FIG. 43. This feature is particularly advantageous because the support arms 106 and barrier portion of the stent 100 can be easily collapsed and folded for easy retrieval into a delivery tube or catheter (e.g. for removing the stent 100 from the lumen or re-positioning the stent 100 to a different site). In particular, the angled position of the barrier arm portions 4300 enable the bather structure 4300 formed by each support arm 106 to fold in towards the longitudinal axis 1800 (e.g. like Christmas tree branches).

FIG. 45 is an end view of a representative embodiment of a stent 100 having 6 support arms 106 (of the type as shown in FIGS. 43 and 44) arranged about a longitudinal axis 1800. The barrier structure 4300 of the support arms 106 form a barrier portion of the stent 100 that surrounds a part of the longitudinal axis 1800. When the stent 100 is configured to the expanded position, as shown in FIG. 42, the barrier structures 4300 of the support arms 106 are also configured to an expanded configuration such that portions of the barrier structure 4300 for one arm 106 may partially overlap with the barrier structure 4300 of other adjacent arms 106. The overlapping configuration of the barrier structures 4300 form the barrier portion of the stent 100. The shape of the barrier structure 4300 of each arm 106 (or the overlapping configuration between the barriers structures 4300 of adjacent arms 106) may define one or more openings that serve the same function as the openings of the barrier members 108 as described above.

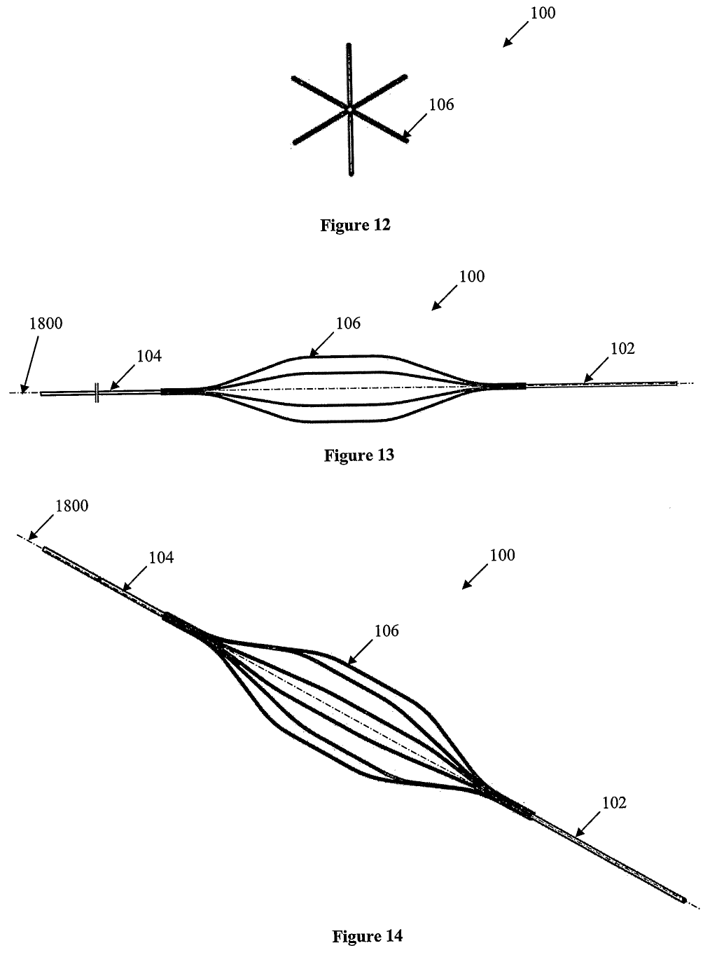

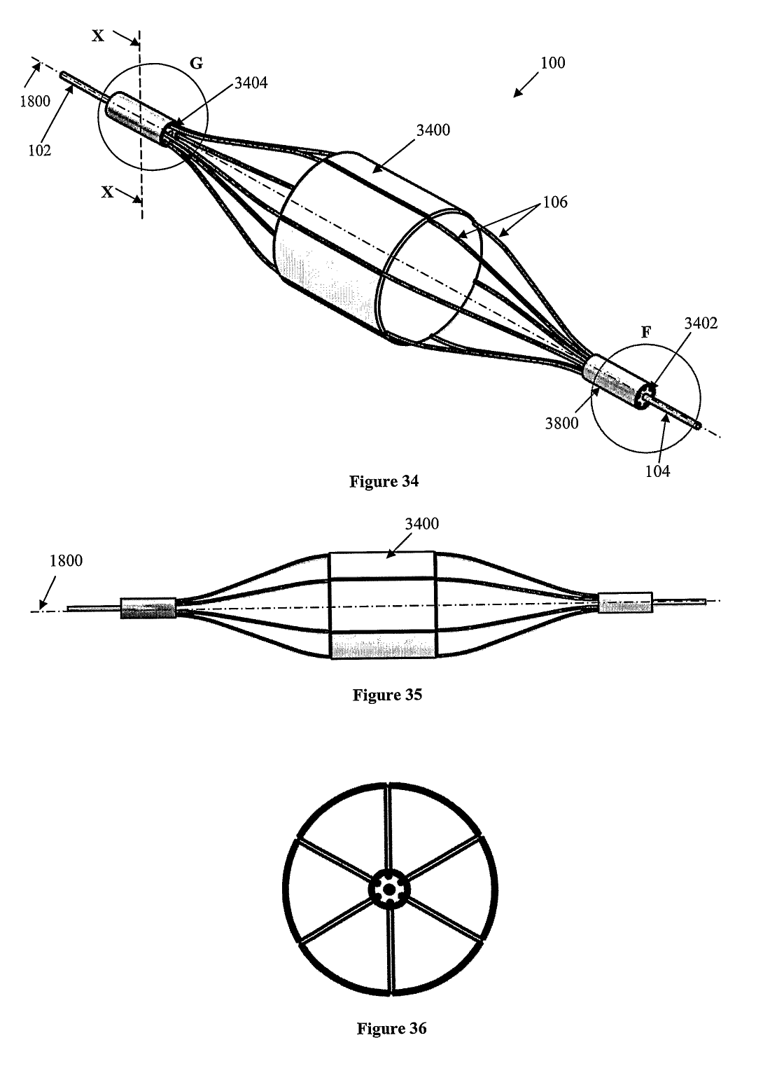

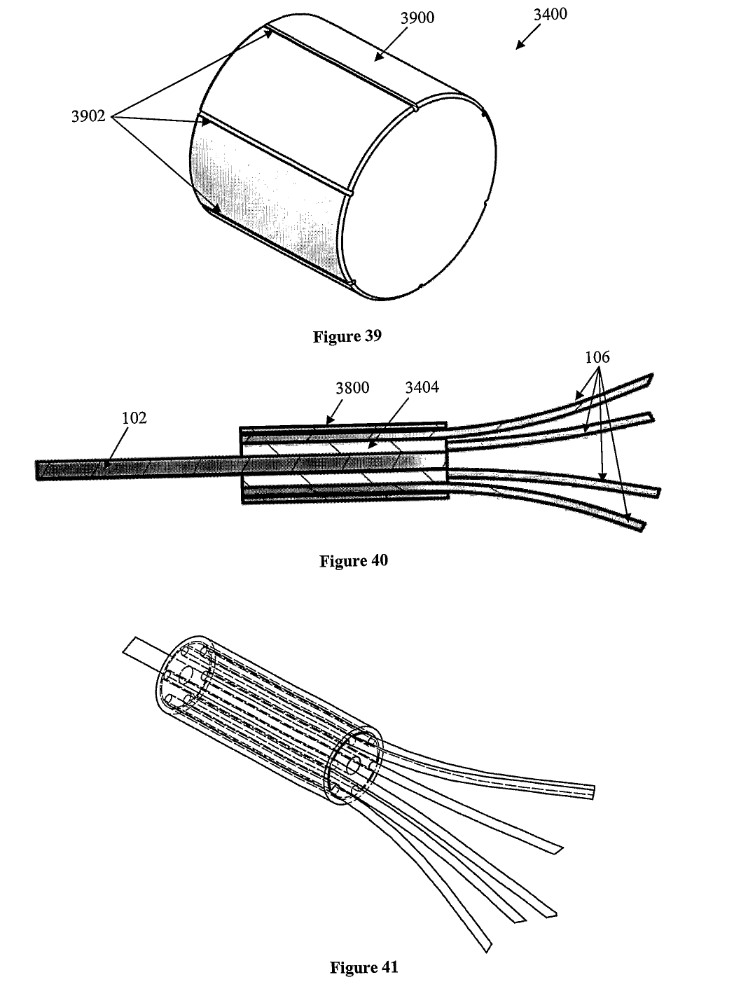

FIGS. 34 to 41 are diagrams relating the assembly of a representative embodiment of a stent 100 as shown in FIGS. 12 and 13 (which has 6 support arms 106). However, it should be understood that the principles described with reference to FIGS. 34 and 41 can be applied in the assembly of any of the representative embodiments of the stent 100.

As shown in FIG. 34, the support arms 106 of the stent 100 are initially held in position by an alignment tool 3400 for evenly spaces the support arms 106 about a longitudinal axis 1800 of the stent 100. FIG. 39 shows an example of the alignment tool 3400, which includes a body 3900 having one or more retaining portions 3902, each for engaging a different support arm 106. Each retaining portion 3902 may, for example, include a groove for receiving a part of a supporting arm 106.

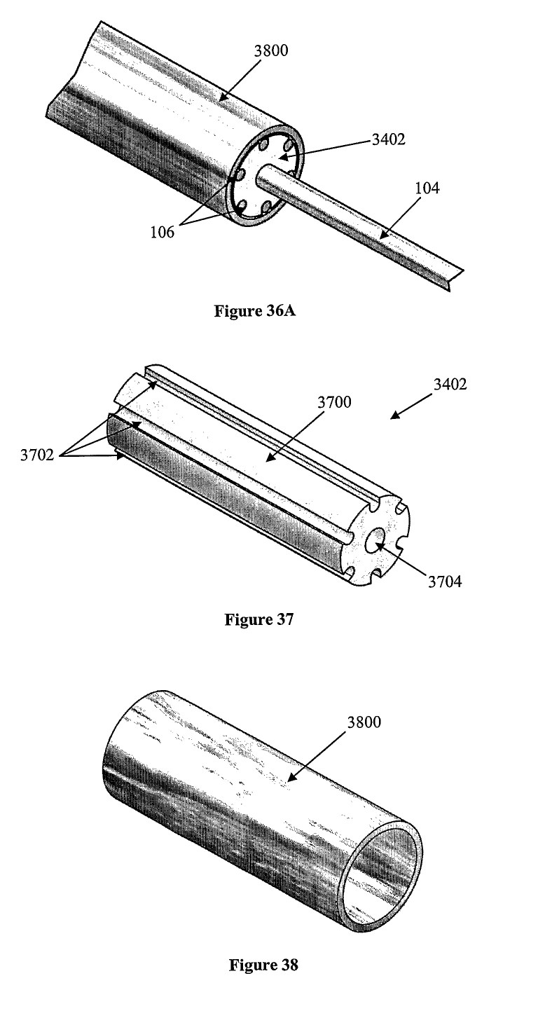

When the support arms 106 are held in position by the alignment tool 3400, the respective ends of the support arms 106 are coupled to the guide portion 102 and drive portion 104 respectively. In a representative embodiment, different ends of the support arms 106 are coupled to a different alignment member 3402 and 3404. FIG. 37 shows an example of one of the alignment members 3402. The alignment member 3402 includes a body 3700 having one or more coupling portions 3702, each being shaped for engaging an end portion of a different support arm 106. The alignment member 3402 may be made from a metallic or plastic material. Each of the coupling portions 3702 may be in the form of a groove (or guide) that is formed on an exterior portion of body 3700 the alignment member 3402.

The alignment member 3402 also includes a connecting portion 3704 for securely engaging either a guide portion 102 or drive portion 104 of the stent 100. For example, the connection portion 3702 may be a hollow shaped for securely receiving either a guide member (of the guide portion 102) or a drive member (of the drive portion 104).

When the support arms 106 are held in position by the alignment tool 3400 and the alignment members 3402 and 3404, a crimping tube 3800 may be used for securely coupling the ends of the support arms 106 to the respective alignment members 3402 and 3404. Alternatively, the ends of the support arms 106 may be securely coupled to the respective alignment members 3402 and 3404 by means of welding, glue or other joining means or techniques.

FIG. 36 is a detailed view of Detail F in FIG. 34, and shows the support arms 106 coupled to the alignment member 3402 in the assembled form. The support arms 106 are coupled to the alignment member 3404 in the same manner. FIGS. 40 and 41 are different views of the coupling arrangement between the support arms 106, alignment member 3404 and crimping tube 3800 corresponding to Detail G in FIG. 34.

The stent 100 is stored inside of a catheter 800 (e.g. a microcatheter) prior to use in a linearly collapsed state (as shown in FIGS. 8, 9 and 10). When stored inside the catheter, the inner wall of said catheter 800 forces the arms 106 closer together in said compressed position. The stent is released from the catheter by pulling a control wire attached to the catheter 800, which results in the displacement of the catheter 800 relative to the arms 106 of the stent 100. As shown in FIGS. 8, 9 and 10, the catheter is being pulled towards the right hand side of each drawing, whilst the stent 100 is released towards the left hand side as a result of the catheter's 800 movement. When the catheter 800 no longer engages the arms 106, the arms 106 automatically adjust to its expanded (or deployed) position.

The stent 100 may include an inflatable balloon (not shown) that, during inflation, forces the arms 106 to move towards the expanded position. For example, to provide further reinforcement of the arms 106, the balloon may be placed on the drive member 104 inside the cavity surrounded by the arms 106 when in the expanded position. The balloon can also be deployed (i.e. inflated) to stop blood from flowing through the lumen 700, such as in the event of a ruptured aneurysm or other emergency.

The stent 100 may serve as an intravascular flow modifier and can provide temporary intravascular reinforcement to blood vessels that are proximate to a cerebral aneurysm. In this way, the stent will serve to divert blood flow away from the ruptured aneurysm whilst repair can be effected. The stent 100 can be used in combination with vaso-occlusive devices placed in a brain aneurysm for the purpose of occluding an aneurysm, whereby the stent 100 provides reinforcement for the area of the blood vessel in the vicinity of the aneurysm. For example, the stent 100 can be placed adjacent to the neck of an aneurysm for access and insertion of embolic coils or other devices in to the aneurysm. The stent 100 can be retrieved following the procedure, and can be later redeployed.

FIG. 10 is a diagram showing a representative embodiment of the stent 100 in use. The microcatheter 800 is maneuvered to place the stent 100 into the desired position in the lumen 700. Upon deployment (i.e. removal of the microcatheter 800 via an over-the-wire or rapid exchange configuration), the stent 100 is placed within the vasculature (i.e. lumen 700) so that it opens and extends from a position proximal to the aneurysm to be treated. The stent 100 may be arranged so that a barrier member 108 forms a permeable barrier that substantially straddles across the neck portion of the aneurysm to allow placement of one or more embolic coils (or another vaso-occlusive device) through the openings of the barrier member 108 and into the aneurysm. During this process, blood inside the lumen is allowed to flow through the lumen and into the aneurysm. This enables the vaso-occlusive devices in the aneurysm to induce thrombosis, which blocks off the aneurysm. Following treatment of the aneurysm, the stent 100 is received back into the microcatheter 800 for removal from the patient's body.

The steps for operating the stent 100 to retain vaso-occlusive devices in an aneurysm involves:

i) positioning the stent 100 adjacent to said aneurysm;

ii) adjusting the arms 106 to their expanded position, which stretches the barrier member 108 to form a permeable barrier adjacent to a neck portion of the aneurysm;

iii) delivering one or more vaso-occlusive devices through the permeable barrier and into the aneurysm (e.g. using delivery catheter 702), so that after the vaso-occlusive devices have been is released into the aneurysm, the barrier retains these devices within the aneurysm and inhibits these devices from moving away from the aneurysm.

The stent 100 may be delivered and left in-situ. The stent 100 may have a weakened area where the drive member (e.g. a push wire) is broken off from the drive portion 104 of the stent 100 to leave the stent 100 in place inside the lumen (e.g. after the stent 100 has been deployed).

The stent 100 may be removed by the drive member reengaging with the drive portion 104 of the stent 100 to pull the stent 100 back into the delivery tube or catheter (e.g. for removal or re-delivery at a different site). The drive member and drive portion 104 may have a portion that is correspondingly shaped for forming a releasable hooking engagement with each other. This allows the drive member to reengage with the drive portion 104 for removing the stent 100 (e.g. by pulling it back into a delivery tube or catheter) or for repositioning the stent 100 in the lumen. Once the stent 100 is fully received into the delivery tube or catheter, the stent 100 can be ejected from the delivery tube/catheter (for redeployment) using a plunger that selectively moves inside the core of the delivery tube/catheter under the control of a user.

The stent 100 is preferably made of nitinol, nickel or titanium alloy, or another alloy with similar elastically deformable or spring-like properties. The stent 100 can be made to different widths and lengths (e.g. when fully expanded).

Advantageously, the stent 100 may be potentially used in arteries up to renal size while still providing the benefits of placement without the use of balloons or mechanical expansions. One significant benefit in such an application is that the flow through the vessel is never fully occluded by the placement of the stent 100, and it is possible to place or deploy the stent 100 from a free flow guiding catheter 800 that is relatively small in diameter compared to the inside diameter of the blood vessel being treated.

While certain features of the invention and its use have been described, it will be appreciated by those skilled in the art that many forms of the invention may be used for specific applications in the medical treatment of deformations of the vasculature. Other features and advantages of the present invention would become apparent from the following detailed description taken in conjunction with the accompanying drawings, which illustrate by way of example, the principles of the invention.

As shown in the exemplary drawings, which are provided for the purposes of illustration and not by way of limitation, the device of the present invention is designed to be deployed intravascularly without the necessity of balloons or other expansive elements and can be deployed from a guiding catheter directly into the area to be treated. The intravascular device of the present invention is particularly useful for treatment of damaged arteries incorporating aneurysms and the like, particularly those which are treatable by the use of embolic coils or other embolic devices or agents used to occlude the aneurysm. More particularly, the device of the invention is particularly well adapted to use with the types of catheters used to place such embolic coils in aneurysms, and the device may be used to reinforce the area in the vicinity of the aneurysm while allowing placement of one or more embolic coils through the gaps in the stent, while assisting in the retention of the embolic devices within the dome of the aneurysm.

The invention provides numerous important advantages in the treatment of vascular malformations, and particularly malformations which include the presence of aneurysms. Since the device does not represent an essentially solid tubular member, and does not require the use of a balloon or other mechanical device for deployment, it is capable of deployment from a guiding catheter which need not occlude the artery as it is put into a position from which to deploy the device. Furthermore, the device upon deployment can reinforce the artery without occluding access to the aneurysm, thus allowing the device to be deployed prior to the placement of embolic coils or the like in the aneurysms. Alternatively, depending on the nature of the vascular defect, the embolic coils or other embolic occlusive or other vasoocclusive devices can be placed and the device deployed thereafter to hold the devices in the aneurysm.

The present invention offers a number of other advantages. For example, the stent 100 is able to provide selective reinforcement in the vicinity of the artery, while avoiding any unnecessary trauma or risk of rupture to the blood vessel, and allows retrieval of the device at the conclusion of the procedure. The stent 100 can also be temporarily deploy (for selective reinforcement device) with continuous blood flow through the lumen 700.

The stent 100 can be used to treat vascular malformations, and particularly ruptured aneurysms in the neurovasculature. Importantly, the stent 100 can be particularly useful when used in combination with vaso-occlusive devices placed in the aneurysm by intravascular procedures.

Modifications and improvements to the invention will be readily apparent to those skilled in the art. Such modifications and improvements are intended to be within the scope of this invention.

Throughout this specification and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. An endovascular stent, including:
a guide portion and a drive portion;
a plurality of flexible support arms, each including two opposing ends that are coupled to said guide portion and said drive portion respectively, said arms being moveable relative to each other between an expanded position and a compressed position; and
said stent including at least one barrier portion, such that when said arms are moved to said expanded position, said arms configure the barrier portion into a selectively permeable barrier through which one or more articles may be introduced into a space between said barrier and a wall of a lumen receiving said stent.

2. A stent as claimed in claim 1, wherein said arms are arranged about a longitudinal axis, said arms being movable away from said axis to said expanded position, and movable towards said axis to said compressed position.

3. A stent as claimed in claim 1, wherein each of said arms has one or more portions arranged parallel to said axis.

4. A stent as claimed in claim 1, wherein said at least one barrier portion is configurable by said arms to define one or more openings through which said articles may be introduced to said space.

5. A stent as claimed in claim 4, wherein the barrier portion is shaped to inhibit movement of said articles away from said space.

6. A stent as claimed in claim 4, wherein said barrier portion includes one or more barrier members coupled to said arms, wherein said openings are formed through at least one of said barrier members.

7. A stent as claimed in claim 4, wherein said barrier portion includes one or more barrier members coupled to said arms, wherein said openings are defined by one or more gaps formed between adjacent said barrier members.

8. A stent as claimed in claim 4, wherein at least some of said arms are shaped for engaging at least a portion of other adjacent said arms when the arms are configured in the expanded position, said openings being defined by one or more gaps formed between adjacent said arms.

9. A stent as claimed in claim 4, wherein at least some of said arms are shaped so as to be selectively adjustable between an expanded configuration and a retracted configuration, such that when said arms are configured in said expanded configuration, the shape of said arm defines one or more of said openings.

10. A stent as claimed in claim 1, wherein said article is a vaso-occlusive device.

11. A stent as claimed in claim 1, wherein each said barrier member includes one or more pockets each for receiving and engaging a portion of said arms to inhibit movement of said barrier member relative to said portion of said arm.

12. A stent as claimed in claim 6, wherein each said barrier member includes a membrane made from at least one of an elastomer, polymer and metallic material.

13. A stent as claimed in claim 12, wherein said membrane includes one or more of the following materials:
i) polytetrafluoroethylene (PTFE),
ii) perfluoroalkoxy polymer resin (PFA),
iii) fluorinated ethylene-propylene (PEP),
iv) fluorinated ethylene-propylene (FEP),
v) polyethylene,
vi) polyurethane,
vii) silicone,
viii) latex,
ix) a rubber including at least one of a natural and synthetic rubber, and
x) an alloy including at least one of nitinol, nickel and titanium.

14. A stent as claimed in claim 1, wherein, when said stent is received in a catheter for storage, the inner wall of said catheter forces said arms towards said compressed position, and when said stent is ejected from said catheter, said arms are biased to move towards said expanded position.

15. A stent as claimed in claim 2, wherein at least a portion of said arms are biased to an angled position relative to said axis so as to enable those portions of said arms to fold towards said axis for storage.

16. A stent as claimed in claim 1, wherein said stent includes an inflatable balloon that, during inflation, forces said arms to move towards said expanded position.

17. A stent as claimed in claim 1, wherein said drive portion is releasably coupled to a flexible drive member for controlling at least one of the lateral movement and positioning of said stent within said lumen, wherein said drive portion is detachable from said drive member when said stent is deployed.

18. A stent as claimed in claim 1, wherein said guide portion includes a flexible guide member for guiding the movement of said stent when pushed along said lumen.

19. A stent as claimed in claim 1, wherein said arms are attached to said guide portion and said drive portion by respective tubular members.

20. A method for introducing a vaso-occlusive device into an aneurysm of a lumen using a stent as claimed in claim 1, including:
i) positioning said stent in said lumen adjacent to said aneurysm;
ii) adjusting said arms to said expanded position to form the permeable barrier having one or more openings adjacent to a neck of said aneurysm; and
iii) delivering said device through the openings into said aneurysm; wherein, after said device is released into said aneurysm, said barrier inhibits movement of said device away from said aneurysm.
